(19) United States
(12) Patent Application Publication
(10) Pub. No.: US 2012/0264817 A1
(43) Pub. Date: Oct. 18, 2012

(54) SOLUBILIZED FORMULATION OF DOCETAXEL WITHOUT TWEEN 80

(76) Inventors: Nageswara R. PALEPU, Southampton, PA (US); Bhanu Teja Bulusu, Hyderabad (IN)

(21) Appl. No.: 13/525,164

(22) Filed: Jun. 15, 2012

Related U.S. Application Data
(63) Continuation of application No. 12/214,506, filed on Jun. 19, 2008.
(60) Provisional application No. 60/936,763, filed on Jun. 22, 2007.

(51) Int. Cl. A61K 31/337; A61P 35/00

(52) U.S. Cl. 514/449

(57) ABSTRACT

Lyophilizates containing docetaxel and the use thereof in preparing concentrated liquid formulations, and ready to use formulations for injection, as well as such concentrates and ready to use formulations themselves are disclosed in which Tween surfactants are avoided so that hypersensitivity reactions to Tween surfactants can be avoided and docetaxel can be administered at higher doses and/or for longer periods of time and/or for additional treatment cycles.

### CROSS-REFERENCE TO RELATED APPLICATIONS

This application is related to pending U.S. 60/936,763, filed Jun. 22, 2007.

### STATEMENT REGARDING FEDERALLY SPONSORED RESEARCH OR DEVELOPMENT

Not Applicable

### FIELD OF THE INVENTION

The present invention relates to a lyophilizate of docetaxel and a method of making the same and to the use thereon in the preparation of (a) an injectable liquid concentrate; (b) injectable aqueous formulations thereof with injectable aqueous carrier fluids, (c) such injectable liquid concentrates; and (d) such liquid formulations, the final dilution formulations having concentrations of the docetaxel suitable for injectable administration, each without the need for polysorbate 80.

### BACKGROUND OF THE INVENTION

Docetaxel is an antineoplastic agent belonging to the taxoid family being marketed by Sanofi-Aventis under trade name Taxotere®. It is prepared by semisynthesis beginning with a precursor extracted from the renewable needle biomass of yew plants. The chemical name for docetaxel is (2R,3S)—N-carboxy-3-phenylisoserine, N-tert-butyl ester, 13-ester with 5beta-20-epoxy-1,2α,4,7β,10β,13α-hexahyciroxytax-11-en-9-one 4-acetate 2-benzoate, trihydrate. Docetaxel has the following structural formula:

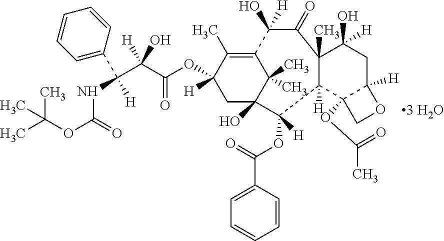

Docetaxel, as currently marketed by Sanofi-Aventis, is a white to almost-white powder with an empirical formula of C₄₃H₅₃NO₁₄.3H₂O, and a molecular weight of 861.9. It is highly lipophilic and practically insoluble in water. Taxotere® (docetaxel) Injection Concentrate is a clear yellow to brownish-yellow viscous solution. Taxotere® is sterile, non-pyrogenic, and is available in single-dose vials containing 20 mg (0.5 ml) or 80 mg (2 ml) docetaxel (on an anhydrous basis). Each ml contains 40 mg docetaxel (on an anhydrous basis) and 1040 mg polysorbate 80. For purposes of this specification, reference to an amount of “docetaxel” without reference to the specific form (i.e., hydrate, salt, etc.) will mean the stated amount of the free, anhydrous, non-solvated moiety of the drug in question unless the context clearly requires otherwise, notwithstanding the actual form of the compound then under discussion. Thus, for example, reference to 80.7 mg of docetaxel without reference to the form of the drug, means that amount of the actual drug form used which corresponds to the same number of moles of the docetaxel moiety as 80.7 mg of free, unsolvated, anhydrous docetaxel. If free docetaxel trihydrate were to be used, this would mean 86.1 mg of free docetaxel trihydrate. Similar calculations for salts and solvates will be apparent to those of ordinary skill in the art.

Taxotere® Injection Concentrate requires dilution prior to use. A sterile, non-pyrogenic, single-dose diluent is supplied for that purpose. The diluent for Taxotere® contains 13% ethanol in water for injection, and is supplied in vials. The preparation of the dilution is in two phases. The concentrate (which is stored between 2-25° C. (36 and 77° F.)) is allowed to come to room temperature, if not already, along with any necessary diluent (13% ethanol in water for injection for the commercially available material) by letting them stand under room temperature conditions for about 5 minutes. Diluent is aseptically withdrawn from its vial (approximately 1.8 ml for Taxotere®20 mg and approximately 7.1 ml for Taxotere® 80 mg) into a syringe by partially inverting the vial, and transferring it to the appropriate vial of Taxotere® Injection Concentrate. If the procedure is followed as described, an initial diluted solution of 10 mg docetaxel/ml will result. This initial dilution is mixed by repeated inversions for at least 45 seconds to assure full mixture of the concentrate and diluent. The vial should not be shaken. The resulting solution (10 mg docetaxel/ml) should be clear; however, there may be some foam on top of the solution due to the polysorbate 80. The initial diluted solution may be used immediately or stored either in the refrigerator or at room temperature for a maximum of 8 hours.

The current Taxotere label indicates that the required amount of docetaxel is then aseptically withdrawn from the initial 10 mg docetaxel/ml solution with a calibrated syringe and injected into a 250 ml infusion bag or bottle of either 0.9% Sodium Chloride solution or 5% Dextrose solution to produce a final concentration of 0.3 to 0.74 mg/ml. If a dose greater than 200 mg of Taxotere® is required, a larger volume of the infusion vehicle is used so that a concentration of 0.74 mg/ml docetaxel is not exceeded. (It has been found that if this maximum is exceeded in the final infusion concentration, the Taxotere® precipitates out of the formulation having the polysorbate as the solubilizer.) The infusion is then thoroughly mixed by manual rotation. The final Taxotere® dilution for infusion should be administered intravenously as a 1-hour infusion under ambient room temperature and lighting conditions.

Taxotere® infusion solution, if stored between 2 and 25° C. (36 and 77° F.) is stable for 4 hours. Fully prepared Taxotere® infusion solution (in either 0.9% Sodium Chloride solution or 5% Dextrose solution) should be used within 4 hours (including the 1 hour intravenous administration).

The present marketed docetaxel (in Taxotere) is dissolved in 100% (w/v) polysorbate 80 (Tween-80) which results in severe side effects. Severe hypersensitivity reactions characterized by generalized rash/erythema, hypotension and/or bronchospasm, or very rarely fatal anaphylaxis, have been reported in patients in spite of receiving the recommended 3-day dexamethasone premedication. Hypersensitivity reactions require immediate discontinuation of the Taxotere® infusion and administration of appropriate therapy. All the hypersensitive reactions mentioned above are primarily caused by and due to the presence of polysorbate 80 in the formulation. In order to reduce the side effects induced by polysorbate 80, all patients are treated with dexamethasone for three days prior to therapy. Dexamethasone is a steroid which suppresses the immune-response in patients. Cancer patients under chemotherapy generally have a low level of immunity due to the destruction of healthy cells by the chemotherapeutic agents. Treatment with steroids will further compromise the patient's immunity and patients will be susceptible to bacterial and fungal attacks. Due to these side effects, most of the patients drop out of docetaxel therapy by the end of 2^{nd} or 3^{rd} cycle or skip a dose or continue further therapy at reduced dose. The recommended therapy is 6 cycles of docetaxel given once every three weeks. Thus, therapeutic activity and the maximum tolerated dose (MTD) of docetaxel are compromised due to the presence of polysorbate 80 in the formulation. Other solubilizing agents such as Cremophor EL (used in connection with the marketed paclitaxel product Taxol®) having similar allergic reactions (requiring pre-medication with steroids and antihistamines) should be avoided.

### OBJECTS OF THE INVENTION

It is therefore an object of the invention to provide a docetaxel formulation suitable for injection with little or no polysorbate 80 surfactant.

It is a further object of the invention to provide a docetaxel formulation suitable for injection with little or no alcohol.

It is another object of the invention to provide a docetaxel formulation suitable for injection having no polysorbate 80 surfactant and no alcohol.

Yet another object of the invention is to provide a docetaxel liquid concentrate formulation that has little or no polysorbate 80 surfactant and further has little or no Cremophor surfactant.

Still another object of the invention is to provide a docetaxel liquid concentrate that has little or no polysorbate.

Another object of the invention is to provide a docetaxel liquid concentrate that has both little or no polysorbate and little or no Cremophor component.

Still another object of the invention is to provide a docetaxel liquid concentrate that is completely free of polysorbate components.

An even further embodiment of the invention is to provide a docetaxel liquid concentrate that is completely free of both polysorbate and Cremophor components.

It is yet another object of the invention to provide a docetaxel formulation that has fewer hypersensitivity reactions than the currently commercially available formulations, which currently available formulations have a polysorbate 80 surfactant component.

It is yet another object of the invention to provide a docetaxel formulation that has fewer hypersensitivity reactions than the currently commercially available formulations, which currently available formulations have a polysorbate surfactant component.

It is yet another object of the invention to provide a docetaxel formulation that has fewer hypersensitivity reactions than the currently commercially available formulations, which currently available formulations have a polysorbate 80 surfactant component and an alcohol component.

Still another object of the invention is to provide a substantially polysorbate-free docetaxel liquid concentrate formulation that is also substantially free of hydroxyalkyl-substituted cellulosic polymers.

An even further object of the invention is to provide a substantially polysorbate-free and substantially Cremophor-free docetaxel liquid concentrate formulation that is free of hydroxyalkyl-substituted cellulosic polymers.

Still another object of the invention is to provide a substantially polysorbate-free docetaxel liquid concentrate formulation that is also substantially free of substituted cellulosic polymers.

An even further object of the invention is to provide a substantially polysorbate-free and substantially Cremophor-free docetaxel liquid concentrate formulation that is free of substituted cellulosic polymers.

Still another object of the invention is to provide a substantially polysorbate-free docetaxel liquid concentrate formulation that is also substantially free of cellulosic polymers.

An even further object of the invention is to provide a substantially polysorbate-free and substantially Cremophor-free docetaxel liquid concentrate formulation that is free of cellulosic polymers.

Still another object of the invention is to provide a suitable primary dilution formulation for use in preparing the aforementioned docetaxel liquid concentrates.

An even further object of the invention is to provide a final dilution for injection of a docetaxel containing product in the substantial absence or in the total absence of polysorbate 80 surfactant.

An even further object of the invention is to provide a final dilution for injection of a docetaxel containing product in the substantial absence or in the total absence of polysorbate 80 and in the substantial absence of Cremophor.

An even further object of the invention is to provide a final dilution for injection of a docetaxel containing product in the substantial absence or in the total absence of polysorbate 80 surfactant, in the substantial or total absence of Cremophor, and in the substantial or total absence of a hydroxyalkyl-substituted cellulosic polymer.

An even further object of the invention is to provide a final dilution for injection of a docetaxel containing product in the substantial absence or in the total absence of polysorbate 80 surfactant, in the substantial or total absence of Cremophor, in the substantial or total absence of a hydroxyalkyl-substituted cellulosic polymer, and in the substantial or total absence of alcohol.

An even further object of the invention is to provide a final dilution for injection of a docetaxel containing product in the substantial absence or in the total absence of polysorbate surfactant.

An even further object of the invention is to provide a final dilution for injection of a docetaxel containing product in the substantial absence or in the total absence of polysorbate and in the substantial absence of Cremophor.

An even further object of the invention is to provide a final dilution for injection of a docetaxel containing product in the substantial absence or in the total absence of polysorbate surfactant, in the substantial or total absence of Cremophor, and in the substantial or total absence of a hydroxyalkyl-substituted cellulosic polymer.

An even further object of the invention is to provide a final dilution for injection of a docetaxel containing product in the substantial absence or in the total absence of polysorbate surfactant, in the substantial or total absence of Cremophor, in the substantial or total absence of a hydroxyalkyl-substituted cellulosic polymer, and in the substantial or total absence of alcohol.

Still another object of the invention is to provide a suitable primary dilution for use in preparing the aforementioned final dilution for injection formulations of docetaxel.

An even further object of the invention is to provide a docetaxel lyophilizate for reconstitution where the lyophilizate is substantially free or totally free of polysorbate 80 surfactant.

Yet another object of the invention is to provide a docetaxel lyophilizate for reconstitution where the lyophilizate is substantially free or totally free of polysorbate 80 surfactant and substantially free or totally free of a cremophor surfactant.

Yet another object of the invention is to provide a docetaxel lyophilizate for reconstitution where the lyophilizate is substantially free or totally free of polysorbate 80 surfactant, substantially free or totally free of a cremophor surfactant, and substantially free or totally free of a hydroxyalkyl-substituted cellulosic polymer.

Yet another object of the invention is to provide a docetaxel lyophilizate for reconstitution where the lyophilizate is substantially free or totally free of polysorbate 80 surfactant, substantially free or totally free of a cremophor surfactant, substantially free or totally free of a hydroxyalkyl-substituted cellulosic polymer, and substantially free of alcohol.

An even further object of the invention is to provide a docetaxel lyophilizate for reconstitution where the lyophilizate is substantially free or totally free of a polysorbate surfactant.

Yet another object of the invention is to provide a docetaxel lyophilizate for reconstitution where the lyophilizate is substantially free or totally free of a polysorbate surfactant and substantially free or totally free of a cremophor surfactant.

Yet another object of the invention is to provide a docetaxel lyophilizate for reconstitution where the lyophilizate is substantially free or totally free of a polysorbate surfactant, substantially free or totally free of a cremophor surfactant, and substantially free or totally free of a hydroxyalkyl-substituted cellulosic polymer.

Yet another object of the invention is to provide a docetaxel lyophilizate for reconstitution where the lyophilizate is substantially free or totally free of a polysorbate 80 surfactant, substantially free or totally free of a cremophor surfactant, substantially free or totally free of a hydroxyalkyl-substituted cellulosic polymer, and substantially free of alcohol.

Still another object of the invention is to provide a lyophilizate of docetaxel that can be reconstituted without the use of polysorbate 80 surfactant in either the lyophilizate or in the diluents for reconstitution.

Yet another object of the invention is to provide a lyophilizate of docetaxel that can be reconstituted without the use of polysorbate 80 surfactant and without the use of Cremophor surfactant in either the lyophilizate or in the reconstitution diluents.

Another object of the invention is to provide a lyophilizate of docetaxel that can be reconstituted without the use of any of polysorbate 80, Cremophor, and a hydroxyalkyl-substituted cellulosic polymer in either the lyophilizate or in the reconstitution diluents.

Still another object of the invention is to provide a lyophilizate of docetaxel that can be reconstituted without the use of any of polysorbate 80, Cremophor, a hydroxyalkyl-substituted cellulosic polymer and alcohol in either the lyophilizate or in the reconstitution diluents.

Still another object of the invention is to provide a lyophilizate of docetaxel that can be reconstituted without the use of a polysorbate surfactant in either the lyophilizate or in the diluents for reconstitution.

Yet another object of the invention is to provide a lyophilizate of docetaxel that can be reconstituted without the use of a polysorbate surfactant and without the use of a Cremophor surfactant in either the lyophilizate or in the diluents for reconstitution.

Another object of the invention is to provide a lyophilizate of docetaxel that can be reconstituted without the use of any of a polysorbate surfactant, a Cremophor, and a substituted cellulosic polymer in either the lyophilizate or in the diluents for reconstitution.

Still another object of the invention is to provide a lyophilizate of docetaxel that can be reconstituted without the use of any of a polysorbate surfactant, a Cremophor, a substituted cellulosic polymer and alcohol in either the lyophilizate or in the diluents for reconstitution.

Yet another object of the invention is to provide formulations, liquid concentrates, lyophilizates, etc. containing docetaxel that are substantially free or totally free of any cellulosic polymer and can be reconstituted or diluted without the use a substantial amount or without the use of any amount of a cellulosic polymer.

Another object of the invention is to provide a means to administer docetaxel to patients without the need for administering dexamethasone or any other steroid and/or without the need to administer an antihistamine prior to the initiation of the docetaxel administration.

Yet another object of the invention is the avoidance of diarrheal side effect accompanying docetaxel administration primarily, if not totally, due to the polysorbate present in currently marketed docetaxel injection products.

An even further object of the invention is to provide a means to administer docetaxel to patients without the need for administering dexamethasone or any other steroid and/or without the need to administer an antihistamine prior to the initiation of the docetaxel administration and without the need for administering dexamethasone or any other steroid or antihistamine during or after the docetaxel administration.

Still further objects of the invention will be appreciated by those of ordinary skill in the art.

### BRIEF SUMMARY OF THE INVENTION

These and other objects of the invention can be achieved by a composition comprising docetaxel and (a) at least one pharmaceutically acceptable solubilizer excipient that can dissolve docetaxel in amounts of at least 55 mg/ml or (b) a mixture of pharmaceutically acceptable hydrotropes that in concert (although not individually) are capable of dissolving docetaxel in amounts of at least 55 mg/ml or (c) mixtures thereof or (d) at least one pharmaceutically acceptable solubilization excipient that can dissolve docetaxel in amounts of at least 55 mg/ml in combination with at least one pharmaceutically acceptable solubilization aid where the solubilization aid does not alone or in combination with other solubilization aids dissolve docetaxel in amounts of at least 55 mg/ml. These docetaxel solutions are either in the pharmaceutically acceptable solubilizer, hydrotropes, or mixtures thereof directly or in water solutions thereof, generally without further solubilization aids, but further such solubilization aids may be included if desired. Each of the solutions of the invention is in the substantial absence of polysorbate 80, if not the total absence of polysorbate 80 and optionally in the substantial absence of or total absence of one or more of a polyethoxylated vegetable oil, a polyethoxylated castor oil, a polyethoxylated partially hydrogenated vegetable oil, a polyethoxylated partially hydrogenated castor oil, a polyethoxylated hydrogenated vegetable oil, a polyethoxylated hydrogenated castor oil, optionally in the substantial absence of or in the total absence of hydroxypropylmethylcellulose (preferably hydroxyalkyl alkylcellulose, more preferably substituted cellulosic polymers), and optionally in the substantial absence of ethanol. Ethanol may be used in the preparation of the lyophilizate, but it is substantially, if not totally removed during the lyophilization process. The avoidance of the polysorbate 80 and Cremophor type solubilizers avoids the hypersensitivity reactions that plague existing formulations of taxanes and allows for the reduction or elimination of steroid and/or antihistamine pre- and/or post treatment. Avoidance of the polysorbate 80 further avoids the diarrheal side effect caused thereby. Each of these allows for better, more effective dosing regimens and better patient compliance with recommended dosings than with the currently marketed taxane injectables.

### BRIEF DESCRIPTION OF THE SEVERAL VIEWS OF THE DRAWING

Not Applicable

### DETAILED DESCRIPTION OF THE INVENTION

The present invention is directed to (a) formulations of docetaxel, (b) concentrates for preparing injectable formulations of docetaxel, (c) docetaxel lyophilizates for reconstituting into such injectable compositions or into such concentrates for further dilution into such compositions; and further to (d) methods of manufacture of each. Methods of treatment of docetaxel treatable conditions with the docetaxel formulations, especially for treatment without the need for steroid pre-treatment or at least a reduction in the amount of steroid pre-treatment as compared to the present methods of administering docetaxel are also part of the invention as is the treatment without the need for antihistamine pre/post-treatment. The formulations, concentrates, lyophilizates, intermediate dilutions, and final administration injectable presentations are substantially free, preferably totally free of polysorbate 80, more preferably substantially free, still more preferably totally free of any polysorbate surfactant.

If docetaxel is formulated with non-toxic pharmaceutically acceptable excipients, it can be administered to cancer patients at much higher doses (greater than the current dosing range of 75 to 100 mg/m²), or higher infusion rates (up to at least 1 mg/ml in 10 to 15 minutes infusion time), for longer exposure to the drug (more than 6 cycles), and/or less than 3 weeks between cycles; and without missing any dosing cycles or dose reduction due to side effects. In other words, if docetaxel is formulated with pharmaceutically acceptable innocuous excipients, it will be better tolerated in cancer patients and would be highly beneficial to them as they can take the medication for a longer period of time without dose interruption and reduction (and therefore potentially higher total and cumulative dose) compared to the current formulation. Longer exposure to the docetaxel maintains the dose density over a longer period in the tumor and thereby helps to better eradicate the cancer cells and minimizes the relapse of the disease. Furthermore, the reduction or elimination of the steroid pre-treatment phase (in common use with the existing marketed docetaxel product) means fewer concerns with immune system depression, drug-drug interactions with other drugs which the patient may be taking, and the avoidance of side effects of steroid administration. Still further, avoidance of the Tween component (polysorbate component) means removal of a substantial cause of the diarrheal and erythema side effects seen with current docetaxel infusions. Finally, with the removal of the polysorbate component and enablement of administration at higher dosages than currently suitable, docetaxel may now be used to treat conditions which it could not previously be used to treat because of the dose limitations imposed by the polysorbate and/or alcohol components of the current TAXOTERE formulation.

For purposes of the present invention, the terms “solubilizer” and “hydrotrope” will have the following definitions: A “solubilizer” is a solvent that is capable of dissolving docetaxel to prepare liquid concentrate in concentrations of at least greater than 55 mg docetaxel per ml of solution in the solvent or in an aqueous solution of the solvent, while a “hydrotrope” is defined as a material that is present in large quantities to solubilize the lipophilic drug (and further prevents the precipitation of docetaxel (or other lipophilic agent in the formulation) when the liquid concentrate is further diluted to lower concentrations)). A hydrotrope solubilizes docetaxel or any such other lipophilic agent and requires large quantities to dissolve the drug, but still does not dissolve the drug to the extent as the solubilizer, but two or more hydrotropes can act synergistically on solubility such that the combination can be used as a “solubilizer” in the context of the present invention (again provided that the docetaxel has a solubility in that synergistic combination of at least 55 mg/ml). In some instances a solubilizer can provide sufficient degree of dissolution that a separate hydrotrope or other solubilization aid is not needed, but this is generally not the case (i.e. a separate hydrotrope is usually desirable). For clarity, if a solvent can be used to yield a solution in the solvent directly or in a water solution thereof of at least 55 mg docetaxel/ml, preferably at least 60 mg/ml of docetaxel or more, it is a “solubilizer” according to the present invention. For example, Tween 80, glycofurol, ethanol, etc. can be classified as solubilizers while TPGS 1000, PEG 400 and propylene glycol are classified as hydrotropes. The concentration of drug in solubilizer varies depending on the lipophilicity of drug. The table below shows a number of solubility studies with docetaxel. Each of the solvents that are reported to be able to dissolve docetaxel to an amount of at least about 55 mg/ml, preferably at least about 60 mg/ml is a “solubilizer” according to the present invention. Those of ordinary skill in the art will know of other suitable materials by either reference to literature or by conducting simple solubility studies such as those indicated in the Examples below. Some of the remaining materials where docetaxel solubility is greater than or equal to 10 mg/ml in the Table below can be seen to be “hydrotropes” according to the definitions of the present invention, with other materials being neither solubilizers nor hydrotropes but having some ability to dissolve docetaxel being “solubilization aids”. The present invention does not use the polysorbates (Tweens) even though they are excellent solubilizers because of their tolerability problems as injectable solution components, and thus, the present invention is an attempt to obtain similar or better results (than the TAXOTERE formulation) without the use of polysorbate surfactants. Some of the tested solvents, such as N-Methyl 2-Pyrrolidone Labrofac, peceol and maisine 35-1 are not used in the parenteral therapy, and are not materials for use in the invention. We have conducted the solubility studies in these excipients to understand how different excipients containing different functional groups are contributing to the solubility of docetaxel. A solubilizer can also act as a hydrotrope (on dilution with infusion fluid) if it is used in the sufficiently large quantities. For example, docetaxel solubility in glycofurol is about 200 mg/ml. When this liquid concentrate is diluted with water to administration concentrations, docetaxel precipitates out. Hence a special diluent is needed to dilute the liquid concentrate to prevent precipitation of docetaxel. If docetaxel is prepared as about a 10 mg/ml solution in glycofurol, it will not precipitate out when diluted with IV fluids to administration concentrations. Thus, by decreasing drug to glycofurol ratio from 200:1 to about 10:1 (20-fold increase in glycofurol level), glycofurol functions as a solubilizer (in the concentrate) as well as a hydrotrope (in the diluted infusion solution concentration. In the table below and the rest of this specification, the terms “solubilizer” and “hydrotrope” will be used with reference to concentrates (both initial and intermediate) unless specifically indicated otherwise or the context so requires.

| | | | |
|---|---|---|---|
| PEG 400 | 10 | mg/ml | Hydrotrope |
| Propylene Glycol | 10 | mg/ml | Hydrotrope |
| 50% PEG 400/50% PG | 15 | mg/ml | Hydrotrope |
| 2% Lutrol in PEG 400 | 15 | mg/ml | Hydrotrope |
| Tween 80 | 60 | mg/ml | Solubilizer |
| Tween 20 | 90 | mg/ml | Solubilizer |
| Glycerol | 1.65 | mg/ml | Solubilization aid |
| Span 80 | 3.5 | mg/ml | Solubilization aid |
| TPGS 1000 | 50 | mg/ml | Hydrotrope |
| Labrofac (Capric triglyceride | 35 | mg/ml | Hydrotrope |
| PEG 4 ester. Macrogol 200) | | | |
| Peceol (Glycerol mono Oleate 40) | 7 | mg/ml | Solubilization aid |
| Maisine 35-1 (Glycerol mono | 10 | mg/ml | Hydrotrope |
| linoleate) | | | |
| Ethanol | 120 | mg/ml | Solubilizer |
| N-Methyl 2-Pyrrolidone | 17.6 | mg/ml | Hydrotrope |
| Benzyl alcohol | 90 | mg/ml | Solubilizer |
| Benzyl benzoate | 13 | mg/ml | Hydrotrope |
| Acetic acid | 60 | mg/ml | Solubilizer |
| l-lactic acid | 6 | mg/ml | Solubilization aid |
| Glycofurol | 200 | mg/ml | Solubilizer |

Even though some of the tested solvents showed very high solubility of docetaxel therein and would allow the manufacture of liquid concentrates, in a number of instances, on dilution with water and other common diluents (for the preparation of injectable products, such as normal saline or 5% dextrose solution), the docetaxel came out of solution. Thus, the mere suitability of a solvent as a solubilizer is not enough to complete the present invention. Behavior upon dilution with suitable injectable diluent solutions (water for injection, saline solutions, or dextrose solution for injection) needs to be explored as well in order to obtain a suitable product. Such further exploration will be within the ability of one of ordinary skill in the art once apprised of the present disclosures.

Notwithstanding the above, the solubilizers for the present invention can be selected (without limitation) from the group consisting of glycofurol, acetic acid, N-β-hydroxyethyl lactamide, and benzyl alcohol. Ethanol, which may be present in certain embodiments deriving from lyophilizations of docetaxel, and/or certain manufacturing and purification procedures of docetaxel is restricted to use as a solvent in those processes and thus a small amount of ethanol may persist in the active agent. In most embodiments, ethanol is not present in any significant amount (typically less than about 2000 ppm, preferably less than about 1000 ppm, more preferably less than about 500 ppm, still more preferably less than about 250 ppm, and most preferably not more than about 200 ppm), and in many embodiments is completely absent. Other solvents (those not acceptable for being present in the final formulation for injection) for docetaxel may be used in the lyophilization process provided they are removed during the lyophilization process, but preferably they are not employed even in the lyophilization procedure. Glycofurol is also known as tetrahydrofurfuryl alcohol polyethylene glycol ether and has the following structure:

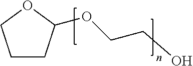

where n is on average 2 for glycofurol 75, but may be other integers for other glycofurols. Glycofurol, especially glycofurol 75, is one of the most preferred solubilizers as docetaxel is highly soluble therein (200 mg/ml in glycofurol 75). While glycofurol 75 is the most preferred of the glycofurols, those having an average n in the above formula of about 2 to about 8, preferably 2 to about 6, more preferably 2 to about 4, more preferably about 2 or about 3 or about 4 are also suitable. Larger values of n can be used, but the appropriateness of the larger glycofurols (average n in excess of about 8) falls off quickly.

Hydrotropes for the present invention are generally selected (without limitation) from the group consisting of polyethylene glycol, especially PEG 400; propylene glycol, Lutrol 2% in PEG (especially in PEG 400); tocopherol compounds, particularly tocopherol-polyethylene glycols, more particularly tocopherol polyethylene glycol diacid (such as succinates, maleates, etc.) esters, especially tocopherol polyethyleneglycol succinates, most preferably tocopherol polyethylene glycol 1000 succinate (TPGS 1000); Labrofac; Peceol; Maisine 35-I; N-methyl-2-pyrrolidone; benzyl benzoate; ethyl carbonate, propylene carbonate, propylene glycol; 1,3-butylene glycol; C₁₋₄alkylesters of C₁₂₋₁₈saturated, mono unsaturated or di-unsaturated fatty acids, especially ethyl oleate; dioxolanes; glycerol formal; dimethylisosorbide, solketal; gentisic acid; and mixtures thereof. Labrofac; Peceol; Maisine 35-I; and N-methyl-2-pyrrolidone are generally not suitable for injectable use and therefore, these materials are least desired to be used, and should be generally avoided. Some mixtures of the hydrotropes will act synergisitically on the solubility of docetaxel such that the combination can be used as the “solubilizer” of the present invention. Confirmation of which combinations of hydrotropes that will act synergistically on solubility so as to be so used as a solubilizer can be done in routine solubility experiments which are totally within the ordinary skill within the art. When such combinations are used in place of a material which is a solubilizer in its own right, the formulation may contain (a) additional amounts of one of the hydrotropes of the synergistic combination or (b) a different hydrotrope or (c) neither, or may further contain a solubilization aid if so desired.

Docetaxel active agent can be dissolved in the solubilizer (solubilizer includes mixtures of hydrotropes that have the requisite solubility of docetaxel therein to qualify the mixture as a solubilizer) alone or in a mixture of the solubilizer and hydrotrope to obtain a clear solution (i.e. initial high concentrate formulation). This can be in the presence or absence of water and preferably is in the absence of water. When the hydrotrope is to be present in the initial high concentrate solution, it is preferably added to the solubilizer first and the docetaxel (either alone or in solution with a solubilizer) is added to the solubilizer/hydrotrope solution, although other orders of addition are suitable as well. These can then be lyophilized and the lyophilizate reconstituted to form concentrates using solvents, hydrotropes, solublization aids selected from the previously set forth group of materials other than those that are specifically indicated as being avoided and other than those that are not compatible with injectable formulations. The initial high concentrate solution can be stored at room temperature or under refrigeration conditions, preferably refrigerated conditions (preferably about 3-8° C.). The concentrate solution is then diluted with a first diluent that contains solubilizer and optionally hydrotrope (whether or not hydrotrope is present in the initial concentrate already) or may be diluted with just injectable diluent fluid alone if the solubilizer/hydrotrope are both already present, or with diluent having one or both of the solubilizer and/or hydrotrope regardless of whether the solubilizer/hydrotrope are otherwise present to obtain an intermediate concentrated solution generally in the concentration range of 5-20 mg docetaxel/ml or higher, preferably about 10 mg/ml (although other intermediate concentrations can be formed as well). This intermediate concentrate is further diluted with an injectable diluent solution (generally water for injection, normal saline solution, or dextrose 5% for injection) to concentrations of 0.3 to 0.74 mg/ml, for administration designed to be in the same concentration range as that recommended in the currently marketed Taxotere® product; however, as discussed earlier, higher infusion concentrations (at least up to 1 mg docetaxel/ml or higher) as well as faster infusion rates are also suitable for the present invention since there is no polysorbate component present. If the hydrotrope is not present in the concentrate formulation, then the diluent solution to prepare the intermediate concentrate should either have the appropriate amount of hydrotrope present or the hydrotrope may be added separately to the concentrate at a point in time before dilution with the injectable diluent solution. If desired, the initial high concentrate solution may be diluted directly by the injectable diluent (normal saline, water for injection, or D5W for example) to achieve the Taxotere® recommended administrable concentration of not more than about 0.74 mg docetaxel per ml (or higher if desired) if the initial high concentration solution has sufficient amounts of both the solubilizer and hydrotrope present, although it is best to prepare the dilution in the two step process set out above. In a highly preferred embodiment, the docetaxel is dissolved in a solubilizer (preferably glycofurol) to a concentration of about 40 mg/ml or higher to form a first concentrate solution. Separately, a hydrotrope (preferably TPGS 1000) is dissolved in a solubilizer (preferably glycofurol)/water mixture to arrive at a hydrotrope concentration of about 215 mg/ml in the solubilizer/water mixture (which is referred to herein as one embodiment of the diluent for the docetaxel concentrate). This liquid concentrate and the diluent solution may then be packaged and stored for commercial distribution. The diluent solution is then used to dilute the docetaxel concentrate to an intermediate concentration of about 5 to about 20 mg docetaxel/ml, preferably about 8 to about 15 mg docetaxel/ml, more preferably about 10 mg docetaxel/ml. The intermediate concentration solution is then diluted to administration concentrations with normal saline, 5% dextrose, or other suitable injection diluents for administration to the patient. In all cases, polysorbate 80 is limited to very minor amounts (substantially free of polysorbate 80), or is completely absent, preferably completely absent; more preferably any polysorbate is substantially absent and most preferably completely absent from the foregoing. In some embodiments, the lyophilizates, liquid concentrates, the intermediate concentrates, and the diluted for administration formulations are substantially free of, more preferably totally free of Cremophor, and preferably substantially free of, still more preferably totally free of all polyethoxylated vegetable oils (whether totally hydrogenated, partially hydrogenated, or not hydrogenated). In other embodiments, the lyophilizates, liquid concentrates, the intermediate concentrates, and the diluted for administration formulations are substantially free of, still more preferably totally free of ethanol. In yet further embodiments, the lyophilizates, liquid concentrates, the intermediate concentrates, and the diluted for administration formulations are substantially free of, preferably totally free of hydroxyalkyl substituted cellulosic polymers (preferably substituted cellulosic polymers, more preferably cellulosic polymers). Still other embodiments are substantially free, if not totally free of each of the aforementioned polysorbates, polyethoxylated vegetable oils (whether hydrogenated in whole or in part or not hydrogenated), substituted cellulosic polymers, and ethanol.

In addition to merely dissolving the docetaxel, the raw docetaxel can be lyophilized and presented as a lyophilizate for reconstitution to a concentrate material (of either the initial high concentrate formulation concentrations or directly to the intermediate concentrate formulations or even directly to the administrable concentrations depending on whether the lyophilizate contains either or both of the solubilizer and/or the hydrotrope in the requisite amounts). The lyophilization procedure can be a routine lyophilization using an appropriate solvent for lyophilization purposes. Insofar as the lyophilization solvent is driven off in the course of the lyophilization procedure, lyophilization may use solvents that are not suitable for parenteral administration, but generally will use suitable materials for parenteral use. The docetaxel solution for lyophilization need not be a solution using a solubilizer or a hydrotrope of the present invention as the solubilizer and hydrotrope may then be added after the lyophile is formed, at any of before, at, or upon reconstitution. However, if desired and the particular solubilizer and/or hydrotrope and/or solubilization aids that remain in the lyophilizate during and through the lyophilization procedure may be added to the docetaxel solution before lyophilization so that the lyophilizate contains the appropriate amounts of docetaxel and optionally one or more solubilizers and/or hydrotropes and optionally one or more solubilization aids of the present invention. In such situations as the lyophilizate contains both the solubilizer and hydrotrope in appropriate amounts, reconstitution with the appropriate amount of injectable diluent solution provides the complete formulation of some embodiments of the present invention. In each case, the lyophilizate, the concentrates made therefrom, the intermediate concentrates made therefrom, and the formulation in the administration concentration are each subject to the independent or concurrent restrictions set forth above with respect to polysorbates, Cremophors, polyethoxylated vegetable oils, hydroxyalkyl substituted cellulosic polymers, substituted cellulosic polymers, cellulosic polymers, and ethanol as stated more fully concerning the formulations made without the use of lyophilization.

Additional components that may be incorporated into the invention formulations include auxiliary aids such protectants against oxidative degradation such as, without limitation, antioxidants and free radical scavengers, such as, without limitation, α-lipoic acid (also known as thioctic acid), sulfa amino acids (such as, without limitation, methionine and cysteine), acetone bisulfite and its alkaline salts, ascorbic acid, among others known in the art as suitable for injection purposes. These optional materials are of value as the TPGS component has the potential of being contaminated with a small amount of peroxide molecules formed during its synthesis, which varies from batch to batch. Incorporation of the protectant or free radical scavenger protects the docetaxel from oxidative and free radical degradive processes that may be caused thereby. When included, the lipoic acid is preferably included in the diluent solution used to dilute the initial concentrate to make the intermediate concentrate, but may be included in the lyophilization vial solution. In a preferred formulation, the lipoic acid is present in the intermediate concentration formulation in an amount up to in general about 50 mg/ml, preferably of about 20 to about 40 mg/ml, more preferably about 20 to about 36.6 mg/ml, still more preferably about 22.5 to about 30 mg/ml, most preferably about 25 mg/ml. Thus, when the intermediate concentrate docetaxel is about 10 mg/ml, and the lipoic acid concentration is about 25 mg/ml, upon dilution to final administration concentration of about 0.3 mg docetaxel/ml in the infusion, the lipoic acid concentration is about 0.75 mg/ml, and on dilution of the intermediate concentrate to the infusion administration concentration of 0.74 mg docetaxel/ml, the lipoic acid concentration is about 1.88 mg/ml. To achieve the 25 mg lipoic acid per ml of intermediate concentrate, 200 mg of lipoic acid needs to be added to the 6 ml of diluent used to prepare the intermediate concentrate from every 2 ml of initial concentrate being diluted (i.e., the diluent for combining with the 40 mg docetaxel/ml concentrate has a lipoic acid concentration of 33.3 mg/ml) or 25 mg of lipoic acid per ml of concentrate needs to be added to the concentrate before dilution to the intermediate concentrate or some combination that achieves the same effective concentration (such as inclusion of appropriate amounts in the pre-lyophilization solution) in the intermediate concentrate. An exemplary diluent composition for diluting 2 ml of the initial concentrate (about 40 mg docetaxel/ml) to the intermediate concentrate (10 mg docetaxel/ml) is, without limitation,

| | | |
|---|---|---|
| TPGS 1000 | 1.5 | g |
| Glycofurol | 1.5 | ml |
| Lipoic acid | 200 | mg |
| Water | 3.0 | ml |
| Total | 6.0 | ml |

In a preferred embodiment, 6 ml of the above exemplary diluents solution is added to every 2 ml of an initial concentrate of 40 mg docetaxel/ml to result in a preferred intermediate concentrate of 10 mg docetaxel/ml, which is then diluted to administration concentrations with infusion suitable fluids. When sulfa amino acids are used in place of or in addition to the lipoic acid, they can be used in amounts generally such that the sum of the lipoic acid and the sulfa amino acid amounts meets the limitations for the lipoic acid above. The remaining alternatives for lipoic acid as set forth above can be used in amounts such that once the formulation is diluted to administration concentrations of docetaxel, the alternative is present in an amount that is suitable for infusions at the resultant concentration AND total infusion dose. These amounts will be known to those of ordinary skill in the intravenous infusion administration art, such as by reference to standard pharmaceutical references as the United States Pharmacopoeia and Remington's Pharmaceutical Sciences.

In addition to the lipoic acid component, and as a means to offset the acidic nature, a buffer can be added such as phosphate buffer (or other suitable buffer, such as without limitation, carbonate/bicarbonate buffer), generally in an amount of about 100-400 mg of phosphate buffer for about each 200 mg of lipoic acid or other acidic oxidative protectant in the formulation. The buffer may also be included in the pre-lyophilization solution, but is preferably added in the reconstitution or dilution steps. The buffer is selected so as to be capable to buffer the intermediate concentrate as well as the final infusion solution to a pH of about 5 to about 7.5, preferably about 5.5 to about 7.2, more preferably about 6 to about 7, most preferably about 6.5 to about 7. Appropriate amounts of the free acid or base used and its conjugate salt to create the buffer will be within the ability of those of ordinary skill in the art. For alkali metal salts of acids, potassium is preferred because due to the TPGS used in the diluents, the potassium ion reduces the infusion viscosity rise caused by the TPGS as compared to sodium ion which tends to increase the TPGS induced viscosity rise. Alternate organic buffer materials include, without limitation, the following materials together with their conjugate salts (which free compound/salt conjugate may form in situ from either the free compound or the conjugate salt being added alone as known in the art of buffer materials) adipic acid, amino acids such as, without limitation, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, etc. Potassium hydroxide or sodium hydroxide, preferably potassium hydroxide, can be used to make final pH adjustments upward. The amount of potassium hydroxide used to bring pH in the region of 5 to 7.5 is preferably 25 to 40 mg, but more or less can be used as appropriate. Hydrochloric acid or additional phosphoric acid can be used as needed to make final pH adjustments downward. Bicarbonate or carbonate salts, especially sodium or potassium salts thereof, most preferably potassium salts thereof, may be used to adjust pH as well.

As the present invention is directed to delivery of docetaxel, once diluted to appropriate injection (especially infusion, most particularly IV infusion) concentrations, it may be administered in appropriate amounts for treating docetaxel responsive conditions known in the art. In addition, since the present invention permits higher doses and concentrations than the currently marketed TAXOTERE, the concentrates and administrable dosage forms thereof made from the present invention are also useful for many of the indications known in the art for docetaxel based on non-clinical data for which the current marketed TAXOTERE formulation is not recommended because of an inability to administer docetaxel at a sufficiently high dose, either acutely or cumulatively. These include, without limitation carcinomas such as colorectal, prostate, pancreatic and liquid tumors like lymphoma and leukemia.

The following examples are presented to exemplify, not limit, the scope of the present invention, which is only limited by the claims appended hereto.

### Example 1

### 1. The Concentrate

| | |
|---|---|
| Docetaxel | 80.0 mg |
| TPGS 1000 | 1900.0 mg |

Method of Preparation:
1. TPGS 1000 is taken in a beaker and heated to about 70° C. to melt completely.
2. Docetaxel is added to this molten TPGS and continued heating for about 15 minutes at 60° C.
3. Then this is allowed to cool at room temperature for dilution studies.
2.

| | |
|---|---|
| Diluent | WFI |

Observations:
1. The concentrate turns waxy and viscous when stored at temperature below the room temperature, i.e., 22° C. To disperse this viscous mass, a large amount of WFI is needed to make the system suitable for subsequent dilution. So the first step of making a 10 mg/ml solution cannot be achieved with this liquid concentrate.
2. To achieve a primary dilution of docetaxel of 10 mg/ml, the above concentrate was heated in a water bath to form a viscous liquid, and then diluted with 8 ml of water for injection. The primary dilution is stable for a period of not less than 8 hours, which was a stability condition stipulated in the innovators product. This solution, when further diluted with NS, achieves the targeted concentration range of 0.3-0.74 mg/ml of docetaxel in the final solution for administration. This solution is stable for a period of 24 hours as opposed to the stability of 4 hours with innovators product. In this example, we have achieved the target concentration of docetaxel for administration without the presence of polysorbate 80.
3. Attempts to lower the quantity of TPGS 1000 in the composition of liquid concentrate resulted in the loss of physical stability (precipitation of drug) either the initial dilution or in final dilution.
4. If water is used as primary and secondary diluent, the concentration of TPGS 1000 must be at least 23.75 parts to one part of docetaxel

### Example 2

To avoid the heating step with the formulation cited in Examplel, this Example lowers the quantity of TPGS 1000 but adds ethanol in the concentrate. Inclusion of ethanol coupled with significant reduction of the amount of TPGS 1000 eliminated the formation of waxy plug during storage.

1. The Concentrate:

| | |
|---|---|
| Docetaxel | 80 mg |
| TPGS 1000 | 200 mg |
| Ethanol | 0.6 ml |

Method of Preparation:
1. TPGS is dissolved in Ethanol
2. To this Docetaxel is added and stirred to obtain a clear solution.

2. Diluent Composition:

TPGS 1000 100 mg/ml in water for injection
1. The concentrate is liquid at room temperature and turned waxy only when stored at 5° C. or below, but turned back to free flowing liquid in 5 minutes when kept at room temperature.
2. During the initial dilution step to get 10 mg/ml, the contents of the vial turned into a thixotropic liquid within the vial. This can be made back into a clear solution either by sonication for about 25 min or by heating for about 10 min. The solution is initially clear, but precipitation occurs within 3 hours.
3. The concentration of TPGS 1000 at the first stage of dilution is 120 mg/ml and docetaxel concentration is 10 mg/ml.
4. The initially diluted solution can be further diluted with NS to get the target concentration of 0.3 to 0.74 mg/ml. This solution is stable for 8 hours. The corresponding TPGS 1000 concentration range is 3.6 to 8.9 mg/ml.

### Example 3

In order to avoid the gelling effect during the dilution of the formulation in Example 2, we prepared a new diluent by adding alcohol and by doubling the TPGS 1000 to 2.0 gm per 10.0 ml.

Diluent Composition:

| | | |
|---|---|---|
| TPGS | 2000.0 | mg |
| Ethanol | 3.0 | ml |
| WFI | qs to 10.0 | ml |

1. Initial dilution stage to get 10 mg/ml was achieved being a clear solution with no precipitate observed for about 6 hours. TPGS 1000 concentration is 220 mg/ml. The ratio of drug to TPGS 1000 to keep docetaxel in solution for at least eight hours s 1:22.
2. The diluted solution of step 1 can be further diluted with NS to get the target range of 0.3 to 0.74 mg/ml. This solution is stable for 24 hours. The corresponding TPGS 1000 range is 6.6 to 16.3 mg/ml.

### Example 4

In the next experiment, we replaced ethanol with glycofurol in the liquid concentrate of formulation in Example 2 and in the diluent of Example 3. Since docetaxel showed better solubility in glycofurol (200 mg/ml) compared to ethanol (120 mg/ml), we substituted glycofurol for ethanol to determine whether this particular system would keep docetaxel from precipitation in the concentrate as well as in the primary and secondary dilution stages.

1. The Concentrate:

| | |
|---|---|
| Docetaxel | 80.0 mg |
| TPGS 1000 | 200.0 mg |
| Glycofurol | 0.5 ml |

2. Diluent Composition:

| | | |
|---|---|---|
| TPGS | 2000.0 | mg |
| Glycofurol | 2.5 | ml |
| WFI | qs to 10.0 | ml |

1. The concentrate is liquid at room temperature and turns waxy only when stored at 5 deg C. or below. But turned back to free flowing liquid in two minutes when kept at room temperature.
2. When initial dilution to get 10 mg/ml is attempted, it is achieved easily as a clear solution. The product is physically stable for about 6 hours.
3. The initially diluted solution was further diluted with NS to get the target concentration range of 0.3 to 0.74 mg/ml. This solution is stable for 24 hours under refrigerated conditions and stable for 6 hours at room temperature.

### Example 5, 6 and 7

Using Phospholipid (Phospholipon 90 G) (or in the case of Example 7A using sorbitol) and ethanol as solvents for lyophilization, we have lyophilized a few batches with the compositions described in the table below:

| | Composition per vial | | |
|---|---|---|---|
| | Docetaxel | TPGS 1000 | Phospholipon 90G |
| Example 5 | 50 mg | — | 50 mg |
| Example 6 | 50 mg | 500 mg | — |
| Example 7 | 50 mg | 500 mg | 50 mg |
| Example 7A | 50 mg | — | — |
| Example 7B | 50 mg | | Sorbitol 500 mg |

Method of Preparation:

A solution of 100 mg/ml of Docetaxel, in ethanol is prepared. TPGS solution is prepared at a concentration of 500 mg/ml in ethanol. Phospholipid stock solution in ethanol is prepared at a concentration of 100 mg/ml. The various vials with the compositions as described herein are lyophilized under the conditions set forth below.

Lyophilization Conditions:
1. Shelf temperature is decreased to −35° C. until the product temperature reaches not more than −30° C. as indicated by the thermocouples introduced in vials. Shelf temperature is maintained at this temperature for about 8 hours.
2. The chamber is evacuated to about 50 milli tons.
3. Then shelf temp is increased such that product temperature reaches 0° C. and then maintained at this temperature for about 10 hours.
4. Finally, the product is dried at 30° C.

The texture of the lyophilized cake is excellent in all three formulations. The lyophilized vials were reconstituted with different diluents for targeting the docetaxel at 10 mg/ml for initial dilution and between 0.3 and 0.74 mg/ml upon subsequent dilution of this initial dilution with NS and observed for the onset time for precipitation.

### Example 8

The lyophilized vial of Example #6 was reconstituted with following diluent for initial dilution to obtain 10 mg/ml of docetaxel and observed for time to onset the precipitation of docetaxel.

Diluent Composition:

1. Lactic acid (88% strength): Glycofurol 1:1

0.75 ml of the reconstituted solution was further diluted with Normal Saline to a final concentration of 0.75 mg/ml. This final diluted sample was also observed for the onset time for precipitation.

Initial reconstituted solution is clear and particle free for more than 96 hours as compared to 8 hours for the innovator sample. Time to onset of precipitation for the final dilution sample was about 8 hours against 4 hours for the innovator product.

The concentration of TPGS 1000 is 100 mg/ml in the first stage of dilution and further diluted to 7.5 mg/ml in the second stage of dilution. The concentration of TPGS 1000 was significantly reduced in the lyophilized formulation over that in non-lyophilized liquid concentrate formulations.

### Example 9

The lyophile of Examples 5-7 can also be reconstituted with lactic acid/glycofurol diluent and the reconstituted solution is clear and particulate free, and stable for at least 4 hours. The final diluted solution is also stable for four hours.

### Example 10

The lyophiles of Examples 5-7 can also be reconstituted with 100-250 mg/ml TPGS 1000 to produce a clear particulate free solution.

### Example 11

The lyophiles of Examples 5-7 can also be reconstituted with straight glycofurol to produce a clear particulate free solution.

### Example 12

The lyophiles of Examples 5-7 can also be reconstituted with straight lactic acid to produce a clear particulate solution.

### Example 13

The lyophiles of Examples 5-7 can also be reconstituted with diluted lactic acid to produce a clear particulate free solution.

### Example 14

The lyophiles of Examples 5-7 can also be reconstituted with a mixture of TPGS and lactic acid to produce a clear particulate solution

### Example 15

The lyophiles of Examples 5-7 can also be reconstituted with a mixture of TPGS and glycofurol to produce a clear particulate solution

### Example 16

The lyophiles of Examples 5-7 can also be reconstituted with different strengths of N-(β-hydroxyethyl)-lactamide solution to produce a clear particulate free solution.

### Example 17

The lyophiles of Examples 5-7 can also be reconstituted with a mixture of TPGS and N-(β-hydroxyethyl)-lactamide to produce a clear particulate solution

### Example 18

The lyophiles of Examples 5-7 can also be reconstituted with a mixture of N-(β-hydroxyethyl)-lactamide and glycofurol to produce a clear particulate solution

### Example 19

The lyophiles of Examples 5-7 can also be reconstituted with a mixture of N-(β-hydroxyethyl)-lactamide, TPGS and glycofurol to produce a clear particulate solution

### Example 20

The lyophiles of Examples 5-7 can also be reconstituted with a mixture of combination of the following solvents to produce a clear particulate solution
1. Ethyl carbonate
2. Propylene glycol
3. Polyethylene glycol 400
4. 1,3-butylene glycol
5. Ethyl Oleate
6. Dioxolanes
7. Glycerol Formal
8. Dimethyl isosorbide
9. Solketal
10. Gentistic acid

### Example 21

We have also explored the direct dilution of the liquid concentrate to 0.74 mg/ml which would be easier for the hospital staff to handle. We prepared a liquid concentrate of docetaxel 10 mg/ml in glycofurol and 7.4 ml of this concentrate was diluted with 99 ml of diluent that contains 20 mg/ml of TPGS 1000 and 9 mg/ml of normal saline. The diluted solution is clear for over a week.

### Example 22

The liquid concentrate in the Example 21 can be prepared with the excipients mentioned in Example 20 and can also be diluted to the desired concentration with the combination of diluents mentioned in the same Examples.

### Example 23

Docetaxel is dissolved in glycofurol to give clear solution having a concentration of 40 mg docetaxel/ml. This initial concentrated docetaxel solution is then diluted with a diluent solution (having 1500 mg of Tocopherol Polyethylene Glycol Succinate 1000 dissolved in 3.0 ml of water and 1.5 ml of glycofurol) in a ratio of 1 ml of the docetaxel solution/3 ml of the diluents solution to give an intermediate concentrate solution having 10 mg docetaxel/ml. The intermediate concentrate is then utilized by dissolving 20 ml of the intermediate concentrate (200 mg docetaxel) obtained by pooling three vials (of the 80 mg/vial presentation) of the intermediate concentration solution (having a relatively small wastage amount) in a 250 ml infusion bag of normal saline or 5% Dextrose for delivery of docetaxel at a concentration of 0.74 mg/ml. Lesser amounts of the intermediate concentrates prepared from either 80 mg/vial liquid concentrate or 20 mg/vial liquid concentrate are dissolved in 250 ml or 100 ml infusion bags for delivery of proportionately lower concentrations.

### Examples 24-29

To a concentrate having 40 mg docetaxel/ml in glycofurol, a diluent is added having the components set forth below in an amount sufficient to result in an intermediate concentrate having 10 mg docetaxel/ml.

| | Example 24 | | Example 25 | | Example 26 | | Example 27 | | Example 28 | | Example 29 | |
|---|---|---|---|---|---|---|---|---|---|---|---|---|
| TPGS 1000 | 1.5 | g | 1.5 | g | 1.5 | g | 1.5 | g | 1.5 | g | 1.5 | g |
| Glycofurol | 1.5 | ml | 1.5 | ml | 1.5 | ml | 1.5 | ml | 1.5 | ml | 1.5 | ml |
| α-lipoic acid | | | 200 | | 200 | | 200 | | 200 | | 200 | |
| Water | 3.0 | ml | 3.0 | ml | 3.0 | ml | 3.0 | ml | 3.0 | ml | 3.0 | ml |

| Buffer | — | K2HPO4 | — | | KH2PO4 | — | — |
|---|---|---|---|---|---|---|---|
| | | 50-200 mg | | | 150-400 mg | | |
| KOH | — | 15-25 | 30 | mg | 20-35 | — | — |

| | | | | | | | |
|---|---|---|---|---|---|---|---|
| glycine | — | — | — | — | 75-150 | mg | — |

| | | | | | | | |
|---|---|---|---|---|---|---|---|
| Alanine | — | — | — | — | — | 90-180 | mg |

### Example 30

Docetaxel is dissolved in glycofurol at a concentration of 10 mg/ml. This solution is directly diluted in IV infusion fluid to obtain a concentration range of 0.3 to 0.75 mg/ml. The solution obtained is stable.

1-55. (canceled)

56. A sterile pharmaceutical formulation for use in treatment of a patient in need thereof, comprising:
(a) docetaxel, or a pharmaceutically acceptable salt thereof, wherein the docetaxel or pharmaceutically acceptable salt thereof is in an amount of about 5 mg/mL to about 20 mg/mL;
(b) glycofurol;
(c) one or more hydrotropes; and
(d) water;
wherein the formulation is substantially free of polysorbates and polyethoxylated castor oil; and
wherein the formulation is precipitate-free at about six hours.

57. The pharmaceutical formulation of claim 56, wherein the docetaxel or pharmaceutically acceptable salt thereof is in an amount of about 10 mg/mL.

58. The pharmaceutical formulation of claim 56, further comprising one or more antioxidants.

59. The pharmaceutical formulation of claim 58, wherein the one or more antioxidants comprises α-lipoic acid.

60. The pharmaceutical formulation of claim 58, further comprising a buffer.

61. The pharmaceutical formulation of claim 56, further comprising a buffer.

62. The pharmaceutical formulation of claim 56, wherein the one or more hydrotropes comprises d-alpha-tocopheryl polyethylene glycol succinate (TPGS), polyethylene glycol (PEG), propylene glycol (PG), or a mixture thereof.

63. The pharmaceutical formulation of claim 62, wherein the one or more hydrotropes comprises TPGS 1000.

64. The pharmaceutical formulation of claim 62, wherein the one or more hydrotropes comprises PEG.

65. The pharmaceutical formulation of claim 62, wherein the formulation requires no further dilution before it is combined with an injectable diluent.

66. The pharmaceutical formulation of claim 65, wherein the injectable diluent is selected from the group consisting of water for injection, saline, 5% dextrose solution, and a mixture thereof.

67. A sterile pharmaceutical formulation for use in treatment of a patient in need thereof, comprising:
(a) docetaxel, or a pharmaceutically acceptable salt thereof, wherein the docetaxel or pharmaceutically acceptable salt thereof is in an amount of about 5 mg/mL to about 20 mg/mL;
(b) ethanol;
(c) one or more hydrotropes; and
(d) water;
wherein the formulation is substantially free of polysorbates and polyethoxylated castor oil; and
wherein the formulation is precipitate-free at about six hours.

68. The pharmaceutical formulation of claim 67, wherein the docetaxel or pharmaceutically acceptable salt thereof is in an amount of about 10 mg/mL.

69. The pharmaceutical formulation of claim 67, further comprising one or more antioxidants.

70. The pharmaceutical formulation of claim 69, wherein the one or more antioxidants comprises α-lipoic acid.

71. The pharmaceutical formulation of claim 70, further comprising a buffer.

72. The pharmaceutical formulation of claim 67, further comprising a buffer.

73. The pharmaceutical formulation of claim 67, wherein the one or more hydrotropes comprises d-alpha-tocopheryl polyethylene glycol succinate (TPGS), polyethylene glycol (PEG), propylene glycol (PG), or a mixture thereof.

74. The pharmaceutical formulation of claim 73, wherein the one or more hydrotropes comprises TPGS 1000.

75. The pharmaceutical formulation of claim 73, wherein the one or more hydrotropes comprises PEG.

76. The pharmaceutical formulation of claim 73, wherein the formulation requires no further dilution before it is combined with an injectable diluent.

77. The pharmaceutical formulation of claim 76, wherein the injectable diluent is selected from the group consisting of water for injection, saline, 5% dextrose solution, and a mixture thereof.
